(19) United States
(12) Patent Application Publication
(10) Pub. No.: US 2017/0044521 A1
(43) Pub. Date: Feb. 16, 2017

(54) BIOMASS EXTRACTS AND METHODS THEREOF

(71) Applicant: Lakeview Nutrition LLC, Chicago, IL (US)

(72) Inventors: Dejian Huang, Chapel Hill, NC (US); Boxin Ou, Franklin, MA (US); Jim Galvin, Chicago, IL (US); Eamonn Byrne, Chicago, IL (US)

(21) Appl. No.: 15/305,253

(22) PCT Filed: Apr. 21, 2015
(86) PCT No.: PCT/US2015/026817

§ 371 Date: Oct. 19, 2016

Related U.S. Application Data
(60) Provisional application No. 61/981,957, filed on Apr. 21, 2014.

(51) Int. Cl. C12N 15/10; C12P 19/30; C12P 19/32

(52) U.S. Cl. CPC C12N 15/1003; C12P 19/32; C12P 19/305

(57) ABSTRACT

The invention provides novel and improved methods that allow effective capture of valuable active ingredients in biomass (e.g., DDGS) at cost-effective commercial scale. The invention also provides novel compositions of active ingredients with unique properties (e.g., nutritional values and enhanced bioavailability).

### PRIORITY CLAIMS AND RELATED PATENT APPLICATIONS

This application claims the benefit of priority from U.S. Provisional Application Ser. No. 61/981,957, filed on Apr. 21, 2014, the entire content of which is incorporated herein by reference in its entirety.

### TECHNICAL FIELDS OF THE INVENTION

The invention generally relates to technologies for utilization of biomass. More particularly, the invention provides novel processes that enable efficient, large scale capture of many valuable components of biomass (such as DDGS), and compositions and uses thereof.

### BACKGROUND OF THE INVENTION

Dried Distillers Grains with Solubles (DDGS) is a by-product of the distillery process. The traditional sources of DDGS were from brewers. More recently, the remarkable growth in US bio-ethanol production from 1.7 billion gallons in 2000 to 15 billion gallons in 2014 (http://ethanolrfa.org/pages/statistics) has greatly increased the supply of DDGS. Currently, the US represents 58% of global bioethanol production with a staggering compound annual growth rate (CAGR) of 16.8%. (http://ethanolrfa.org/pages/World-Fuel-Ethanol-Production) Moreover, the US Department of Energy Roadmap requires 40 billion gallons of bio-ethanol by 2030.

More than 95% of all DDGS is now produced by ethanol fuel plants since the predominant feedstock in the US is maize (corn). Between 32 and 39 million tons of DDGS are produced each year in the U.S. and Canada alone, which number is expected to continue to grow. Over 75% of DDGS is used as livestock feed in Canada and the U.S. (“DDGS Overview.” University of Minnesota Department of Animal Science. http://www.ddgs.umn.edu/overview.htm.)

Unlike Wet Distillers Grains (WDG), which has a shelf life of four to five days due to the water content, DDGS have an almost indefinite shelf life and may be shipped to any market regardless of its proximity to an ethanol plant. Corn based distillers grains from the ethanol industry are commonly sold as a high protein livestock feed.

Nutrient compositions of DDGS depend on the sources and quality of grain used and the specific processes that generated the DDGS. Most of the ethanol produced in the U.S. is made from corn. Because corn contains about two-thirds starch and most starch is converted to ethanol during fermentation, the nutrient (e.g., protein, fat, fiber, ash and phosphorus) content of DDGS are 2 to 3 times more concentrated than in corn. There can be a large variation in the nutrient content and quality of DDGS produced in different plants.

Besides corn, wheat, barley, rye and sorghum (milo) may also be used in alcohol production. DDGS from wheat has much higher protein and much lower fat content than distillers products from corn and sorghum.

A major challenge has been to better utilize the large amounts of biomass generated from ethanol plants and brewers. To reduce production cost of fuel ethanol, an urgent need exists for novel and improved utilization of DDGS and other co-products from the bio-refining process. While efforts to extract proteins and biodiesel from DDGS have been reported, there has been no success in capturing many other useful components in DDGS at industrial scale.

### SUMMARY OF THE INVENTION

The invention is based, in part, on the discovery of novel and improved technologies that allow the effective capture of valuable active ingredients of biomass, such as DDGS, at cost-effective commercial scale. Active ingredients that can be efficiently captured include, for example, vitamins, flavonoids, carotenoids, tocopherols, and lipophilic phenolics, phenolic acids and nucleotides. The extract compositions of the invention present a set of active ingredients in unique proportions, such as enhanced bioavailability.

In one aspect, the invention generally relates to a process for extracting one or more active ingredients from a biomass. The process includes: (a) contacting the biomass with a solvent under a condition and for a time sufficient to extract the one or more active ingredients from the biomass into the solvent, thereby giving rise to a residual biomass and a liquid phase comprising the solvent and the extracted one or more active ingredients; (b) separating the residual biomass and the liquid phase comprising the solvent and one or more active ingredients; and (c) removing the solvent from the liquid phase to yield an extract composition comprising the one or more active ingredients as a concentrated mixture or in substantially pure forms.

In another aspect, the invention generally relates to a composition comprising one or more active ingredients extracted by a process of the invention.

In yet another aspect, the invention generally relates to a biomass extract comprising, by weight: from about 0.1% to about 10% of vitamins; from about 0.1% to about 10% of flavonoids; from about 0.1% to about 10% of carotenoids; from about 0.1% to about 10% of tocopherols; from about 0.1% to about 30% of lipophilic phenolics, and from about 0.1% to about 30% of phenolic acids.

In yet another aspect, the invention generally relates to a process for extracting nucleotides from a biomass. The process includes: (a) contacting the biomass with an alkaline aqueous solution under a condition and for a time sufficient to extract nucleic acids from the biomass, thereby giving rise to a remaining biomass and an alkaline aqueous phase comprising the extracted nucleic acids; (b) separating the remaining biomass and the alkaline aqueous phase comprising the extracted nucleic acids; (c) treating the alkaline aqueous phase comprising the extracted nucleic acids to precipitate nucleic acids from the aqueous phase; and (d) separating the precipitated nucleic acids from the aqueous phase to yield an extract composition comprising nucleic acids.

In yet another aspect, the invention generally relates to a composition comprising nucleic acids produced by a process of the invention.

In yet another aspect, the invention generally relates to a composition comprising 5′-nucleotide monophosphate monomers produced by a process of the invention.

In yet another aspect, the invention generally relates to a biomass extract, comprising by weight: from about 0.1% to about 50% of GMP; from about 0.1% to about 50% of UMP; from about 0.1% to about 50% of IMP; and from about 0.1% to about 50% of CMP.

In yet another aspect, the invention generally relates to a composition comprising zein produced by a process disclosed herein. In certain embodiments, zein so produced is similar or substantially identical to zein obtained from a commercial source or extracted from corn.

### BRIEF DESCRIPTION OF THE DRAWINGS

FIG. 1A schematically depicts an exemplary embodiment of the invention relating to extraction of biomass to obtain an extract composition.

FIG. 1B schematically depicts an exemplary embodiment of the invention relating to extraction of biomass to obtain an extract composition.

FIG. 2 schematically depicts an exemplary embodiment of the invention relating to extraction of biomass to obtain an extract composition.

FIG. 3. UV-VIS spectra of salt water extracts of DDGS.

FIG. 4. HPLC analysis of DDGS oil with bioactives (detection wavelength: 300 nm).

FIG. 5. SDS-PAGE image of zeins. Lane 1: commercial zein, 2, zein sample extracted from DDGS; 3, zein from corn. Concentration: 30 mg/mL zein in 70% EtOH was diluted to 10 mg/mL with sample buffer.

FIG. 6. HPLC chromatogram of nucleotide extracts (detection wavelength at 254 nm). Individual concentration of nucleotides are UMP, 4.61±0.05 mg/g; GMP, 2.98±0.04 mg/g, AMP, 3.02±0.04 mg/g.

FIG. 7. Absorption of carotenoids in plasma of mice.

FIG. 8. Absorption of gamma- and alpha-tocopherols in plasma of mice.

FIG. 9. Absorption of phenolics in plasma of mice.

### DEFINITIONS

The term “biomass”, as used herein, refers broadly to any biological material derived from living, or recently living organisms. Biomass can refer to plants or plant-based materials including woody biomass and agricultural biomass. Examples of biomass include corn syrup, corn oil, molasses, silage, agricultural residues (corn stalks, grass, straw, grain hulls, bagasse, etc.), Distillers Wet Grains (DWG), Distillers Dried Grains (DDG), Distillers Dried Solubles (DDS), Condensed Distillers Solubles (CDS), Distillers Dried Grains with Solubles (DDGS), modified DDGS, woody materials (wood or bark, sawdust, timber slash, and mill scrap), poplars, willows, Eucalyptus, switchgrass, alfalfa, prairie bluestem, algae, including macroalgae, etc.). Examples of grain starch include: whole wheat flour, whole oats/oatmeal, whole grain corn/corn meal, brown rice, whole rye, whole grain barley, whole faro, wild rice, buckwheat, triticale, millet, quinoa, sorghum. Examples of starchy vegetables include: parsnip, plantain potato, pumpkin, acorn squash, butternut squash, green peas.

Exemplary biomass also include cellulosic material, lignocellulosic material, hemicellulosic material, carbohydrates, pectin, starch, inulin, fructans, glucans, corn, sugar cane, grasses, switchgrass, sorghum, high biomass sorghum, bamboo, algae and material derived from these. Biomass also includes processed or spent biomass, for example, after fermentation to produce alcohol or other fermentation products.

The terms “fermentation” or “fermenting”, as used herein, refer to the process of transforming an organic molecule into another molecule using a microorganism or group of microorganisms in or on a suitable medium for the microorganisms. The microorganisms can be growing aerobically or anaerobically. For example, “fermentation can refer to transforming sugars or other molecules from biomass to produce alcohols (e.g., ethanol, methanol, butanol); organic acids (e.g., citric acid, acetic acid, itaconic acid, lactic acid, gluconic acid); ketones (e.g., acetone), amino acids (e.g., glutamic acid). Thus, fermentation includes alcohol fermentation.

Fermenting can be accomplished by any organism suitable for use in a desired fermentation step, including, but not limited to, bacteria, fungi, archaea, and protists. Suitable fermenting organisms include those that can convert mono-, di-, and trisaccharides, especially glucose and maltose, or any other biomass-derived molecule, directly or indirectly to the desired fermentation product (e.g., ethanol, butanol, etc.). Suitable fermenting organisms include, for example, yeast or filamentous fungi. The yeast can include strains from a Pichia or Saccharomyces species. In some embodiments, the yeast can be Saccharomyces cerevisiae. In some embodiments, the fermenting is effected by bacteria. In some embodiments, the microorganism (e.g., yeast or bacteria) can be a genetically modified microorganism.

The terms “pre-treatment” or “pre-treating”, as used herein, refer to any mechanical, thermal, biochemical or chemical process, or combination thereof, that render the biomass more susceptible to extraction with a solvent such as alcohol or aqueous alkaline solution.

The term “bioavailability”, as used herein in the context of nutrition and nutritional ingredients, can be defined as the proportion of the administered substance capable of being absorbed and available for use or storage. Thus, bioavailability refers the fraction of a nutrient that is digested, absorbed and metabolized through normal pathways. (Srinivasan 2001 “Bioavailability of Nutrients: A Practical Approach to In Vitro Demonstration of the Availability of Nutrients in Multivitamin-Mineral Combination Products”. The Journal of Nutrition 131 (4 Suppl): 1349S-50S.)

The term “nucleic acid”, as used herein, refers to a polymer of any length, e.g., greater than about 2 bases, greater than about 10 bases, greater than about 100 bases, greater than about 500 bases, greater than 1,000 bases or more bases composed of nucleotides, e.g., deoxyribonucleotides or ribonucleotides. A nucleic acid may exist in a single stranded or a double-stranded form. A double stranded nucleic acid has two complementary strands of nucleic acid may be referred to herein as the “first” and “second” strands or some other arbitrary designation.

The term “nucleotide”, as used herein, refer to nucleoside monophosphate, a sub-unit of a nucleic acid (whether DNA or RNA or analogue thereof), which includes a phosphate group, a sugar group and a heterocyclic base, as well as analogs of such sub-units.

### DETAILED DESCRIPTION OF THE INVENTION

The invention provides novel and improved methods that allow effective and successive capture of valuable active ingredients in biomass (e.g., DDGS) at cost-effective, commercially viable scale. Active ingredients that can be efficiently captured include, for example, vitamins, flavonoids, carotenoids, tocopherols, and lipophilic phenolics, phenolic acids, nucleotides, and zein. The invention also provides novel compositions of active ingredients with unique properties (e.g., nutritional values and enhanced bioavailability). Additionally, the invention reduces cost of biofuel production through efficient and cost-effective utilization of biomass.

Efficient and cost-effective recovery of active ingredients from DDGS, when performed at large scale (Kg scale or greater), encounters a number of challenges. These challenges can become more significant when the scale is at 100 Kg or greater. Typical difficulties include foaming, separation of solid-liquid phases, low extraction yields of desired components. For example, foaming is a common difficulty in large-scale extraction can greatly reduce extraction efficiency and increase operation cost. Also, solid-liquid separation is another common problem that can lead to complicated and time consuming separation procedures involved, which add to production costs. Unique issues also arise in extraction of active ingredients from grain-based DDGS. For example, significant oil contents in the DDGS may reduce the extraction yields and increase the production cost with the need of repeated extractions because, at large scale extraction process, elimination of repeated operation by increasing the extraction efficiency is highly desired as it can greatly reduce the production cost. This can be done by selecting the optimal extraction temperature, time, extraction solvent, application of enzyme to break down nucleic acids into extractable monomers, best ways of solid-liquid separation methods.

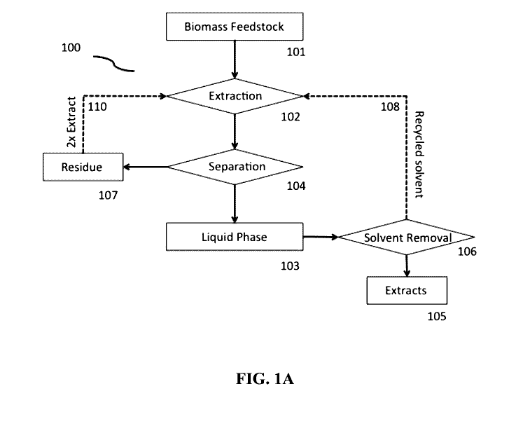

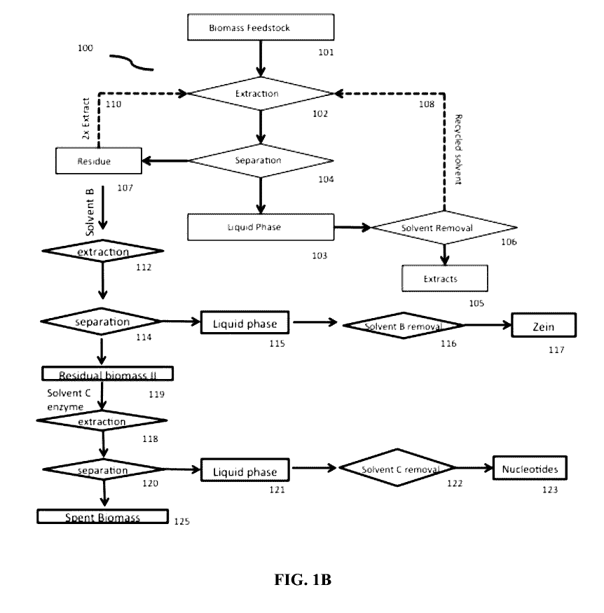

Referring to FIG. 1A, which depicts a flow chart (100) describing an exemplary embodiment of the invention for extracting active ingredients from biomass. Biomass feedstock (101) is fed into an extraction vessel and mixed with a solvent (or solvents), such as water or ethanol, under a select ratio of solvent to biomass. The extraction vessel may be closed or open as required. The extraction (102) takes place under a designed condition of temperature, pressure and length of time. Once the extraction is deemed completed, a separation step (104) takes place whereby the solid and liquid phases are separated. The solid phase or residual biomass (107) may be re-extracted with a fresh solvent (or solvents) to undergo further extraction (110). The liquid phase (103) is allowed to undergo solvent removal (106), which yields crude extract (105). The removed solvent (108), such as ethanol, may be recycled for use in the extraction (102) or other purposes. The crude extract (105) may be used as is or may undergo further treatment and/or purification procedure. In the case of extraction of nucleotides, enzyme is added to the mixture of the water and DDGS to depolymerize nucleic acids to nucleotides to afford nucleotide extracts.

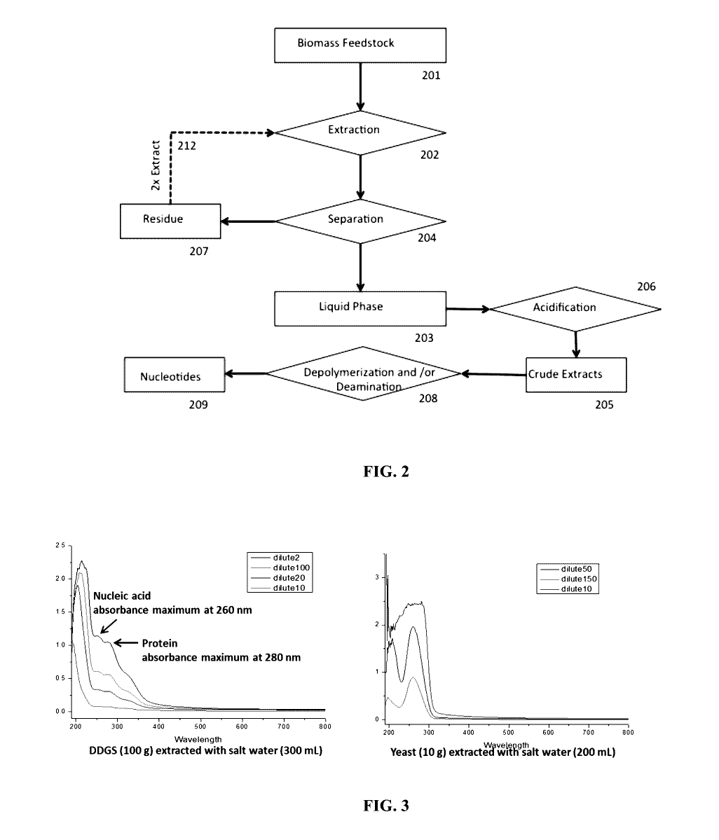

Referring to FIG. 2, which depicts a flow chart (100) describing another exemplary embodiment of the invention for extracting nucleic acids and nucleotides from biomass. Biomass feedstock (201) is fed into an extraction vessel and mixed with a solvent (or solvents), such as aqueous alkaline solution, under a select ratio of solvent to biomass. The extraction vessel may be closed or open as required. The extraction (202) takes place under a designed condition of temperature, pressure and length of time. Once the extraction is deemed completed, a separation step (204) takes place whereby the solid and aqueous phases are separated. The solid phase or residual biomass (207) may be re-extracted with a fresh solvent (or solvents) to undergo further extraction (212). The liquid phase (203) is allowed to undergo acidification and/or addition of other solvents to cause precipitation of nucleic acids, which is then separated to yield a crude extract (205).

In one aspect, the invention generally relates to a process for extracting one or more active ingredients from a biomass. The process includes: contacting the biomass with a solvent A under a condition and for a time sufficient to extract the one or more active ingredients from the biomass into the solvent A, thereby giving rise to a residual biomass I and a liquid phase comprising the solvent A and the extracted one or more active ingredients; separating the residual biomass I and the liquid phase comprising the solvent A and the extracted one or more active ingredients; and removing the solvent A from the liquid phase to yield an extract composition comprising the one or more active ingredients as a concentrated mixture or in substantially pure forms.

In certain preferred embodiments, the process further includes: contacting the residual biomass I with a solvent B to extract proteins into the solvent B, thereby giving rise to a residual biomass II and a liquid phase comprising the solvent B and proteins; and separating the residual biomass II and the liquid phase comprising the solvent B and the extracted proteins; and removing the solvent B from the liquid phase to yield an extract composition comprising proteins.

In certain preferred embodiments, the process further includes: contacting the residual biomass II with solvent C and 5′-phosphodiesterase to extract nucleotides into the solvent; and separating the residual biomass II and the liquid phase comprising the solvent C and the extracted nucleotides; and removing the solvent C from the liquid phase to yield an extract composition comprising nucleotides.

Referring again to FIG. 1B, an exemplary process according to the invention, may include the following steps: (a) contacting the biomass with a solvent (e.g., ethyl acetate, ethanol or a combination thereof), under a condition and for a time sufficient to extract the one or more active ingredients from the biomass into the solvent, thereby giving rise to a residual biomass I and a liquid phase comprising the solvent and the extracted one or more active ingredients; (b) separating the residual biomass I and the liquid phase comprising the solvent and one or more active ingredients; (c) removing the solvent from the liquid phase to yield an extract composition comprising the one or more active ingredients as a concentrated mixture or in substantially pure forms; (d) contacting the residual biomass I with 70% ethanol to extract alcohol soluble protein (zein); (e) separating the residual biomass (which is designated as residual biomass II) and the liquid phase comprising the solvent and zein; (f) removing the solvent from the liquid phase to yield an extract comprising of zein; (g) repeating step (d) to step (f) to maximize the yield of zein; (h) contacting the residual biomass II with water and 5′-phosphodiesterase to hydrolyze nucleic acid to extractable nucleotides; (i) separating the residual biomass (which is designated a spent DDGS) and the liquid phase comprising of nucleotides and other water soluble compounds; (j) removing the solvent from the liquid phase to yield an nucleotide rich fraction; and (k) drying the spent DDGS to produce spent DDGS powder.

Any suitable biomass feedstock may be used. Exemplary biomass feedstock includes fermentation products of plants or plant-based materials. In certain embodiments, the biomass feedstock is fermentation products of various grains (e.g., corn, rice, wheat, barley and rye). In certain preferred embodiments, the biomass feedstock is DDGS. In certain preferred embodiments, the biomass feedstock is DDGS produced from corn-based ethanol fermentation.

In certain embodiments, the biomass may include a spent biomass material from an alkaline aqueous extraction of fermentation product of plants or plant-based materials, for example, a spent biomass material from an alkaline aqueous extraction of fermentation product of grains selected from corn, rice, wheat, barley and rye. In certain embodiments, the biomass may include a spent biomass material generated from an alkaline aqueous extraction of dried distillers grain with solubles (DDGS).

The biomass may be in any suitable form, for example, typically in particulate forms having sizes from about 0.5 μm to about 10 mm (e.g., from about 0.5 μm to about 8 mm, from about 0.5 μm to about 6 mm, from about 0.5 μm to about 5 mm, from about 0.5 μm to about 2 mm, from about 0.5 μm to about 1 mm, from about 1 μm to about 10 mm, from about 10 μm to about 10 mm, from about 50 μm to about 10 mm, from about 1 mm to about 10 mm).

Any suitable solvent (including solvent mixtures) may be used for extraction. In certain embodiments, an alcohol is employed as the solvent. In certain preferred embodiments, ethanol is employed as the solvent.

In certain embodiments, the solvent may include two or more co-solvents, for example, a first or primary co-solvent and a second or secondary co-solvent. In certain embodiments, ethanol is employed as the primary co-solvent and a co-solvent (e.g., another alcohol, water, acetone, ethyl acetate, and hexanes) is also used. The weight ratio of the first, primary co-solvent to the second, secondary co-solvent may be any suitable ratio, for example, from about 5:1 to about 20:1 (e.g., from about 7:1 to about 20:1, from about 10:1 to about 20:1, from about 15:1 to about 20:1, from about 5:1 to about 10:1, from about 5:1 to about 12:1, from about 5:1 to about 15:1, from about 7:1 to about 12:1, from about 7:1 to about 10:1). In certain preferred embodiments, the first co-solvent is ethanol.

In certain embodiments, solvent A is selected from alcohol, water, acetone, ethyl acetate, and hexanes, and combinations of two or more thereof, solvent B is selected from water, ethanol, isopropanol, and fusel alcohol, and combination of two or more thereof; and solvent C is selected from water and salt water.

The weight ratio of the solvent to the biomass may be any suitable ration, for example from about 2:1 to about 15:1 (e.g., from about 2:1 to about 12:1, from about 2:1 to about 10:1, from about 5:1 to about 15:1, from about 5:1 to about 11:1, from about 5:1 to about 9:1, from about 7:1 to about 15:1, from about 7:1 to about 12:1, from about 7:1 to about 10:1, from about 7:1 to about 9:1).

For the step of separating the residual biomass and the liquid phase comprising the solvent and one or more active ingredients, it may be carried out by any suitable technique, for example by filtration and/or centrifugation. The separation step may include a round of filtration or centrifuge or may include two or more rounds of filtration or centrifugation or a combination thereof.

Once the residual biomass is separated from the liquid phase that has the solvent and one or more active ingredients the solvent from the liquid phase is removed, which yields the crude extract of one or more active ingredients.

The solvent may be removed by a variety of techniques, for example, by evaporation, distillation, vacuum transfer, and filtration. Evaporation can be conducted under a raised temperature and/or a reduced pressure. Temperatures and pressures suitable for solvent removal may be selected dependent on the nature of the solvent, the scale of production, whether the recovered solvent is to be recycled and reused in extraction, etc. Generally, evaporation may be effectively carried out at a temperature from about 20° C. to about 100° C. (e.g., from about 30° C. to about 100° C., from about 40° C. to about 100° C., from about 50° C. to about 100° C., from about 60° C. to about 100° C., from about 20° C. to about 100° C., from about 20° C. to about 100° C., from about 20° C. to about 100° C., from about 20° C. to about 100° C.) and at a pressure from about atmospheric pressure to about 1 mmHg. In certain embodiments, the removed solvent from the liquid phase is recycled and used in the extraction step.

Depending on the source of the biomass, a variety of compounds can be extracted according to the processes disclosed herein. Preferably, the one or more active ingredients are selected from vitamins, flavonoids, carotenoids, tocopherols, and lipophilic phenolics, and phenolic acids.

In certain embodiments, the vitamins that may be extracted by the processes herein include one or more of: vitamin E, vitamin B (e.g., vitamin B1, B2, B3, B4, B6), vitamin D, vitamin A.

In certain embodiments, the flavonoids that may be extracted by the processes herein include one or more of: anthocyanins (including both sugar-free anthocyanidin aglycones and anthocyanin glycosides).

In certain embodiments, the carotenoids that may be extracted by the processes herein include one or more of: beta-carotene, lutein, and zeaxanthin.

In certain embodiments, the tocopherols that may be extracted by the processes herein include one or more of: alpha-tocopherol, delta-tocopherol, and gamma-tocopherol.

In certain embodiments, the lipophilic phenolics that may be extracted by the processes herein include one or more of: ferulic acid and its esters and coumaric acid and its esters, caffeic acid and its esters, and synapic acid and its esters.

Depending on the source of biomass and particular extraction conditions, the relative yields of active ingredients may vary, which can be utilized to control the compositions of the resulting extract. For instance, corn-based DDGS usually are higher in carotenoids than wheat-based DDGS.

The process of the invention enables effective recovery of active ingredients. Actual yield of recovery of a particular ingredient depends on factors such as source of biomass, solvent choice, ratio to biomass, temperature and length of extraction, etc. The process can be designed to be suitable for extracting one or more specific active ingredients or class(s) of compounds.

In certain embodiments, the recovery yield for vitamins is 1% or greater, for example from about 20% to 95%, preferably from about 50% to 95%, more preferably from about 70% to about 95%, and most preferably about 90% to about 100%.

In certain embodiments, the recovery yield for carotenoids, is 1% or greater, for example from about 20% to 95%, preferably from about 50% to 95%, more preferably from about 70% to about 95%, and most preferably about 90% to about 100%.

In certain embodiments, the recovery yield for lipophilic phenolics, is 1% or greater, for example from about 20% to 95%, preferably from about 50% to 95%, more preferably from about 70% to about 95%, and most preferably about 90% to about 100%.

The process can also be designed to be suitable for result in extracts of specific combinations of active ingredients or class(s) of compounds. In certain embodiments, for example, the process achieves a recovery yield of 60% or greater yield for vitamins, 60% or greater yield for carotenoids, and 60% or greater yield for lipophilic phenolics. In certain embodiments, the process achieves a recovery yield of 60% or greater yield for vitamins, 60% or greater yield for carotenoids, and 60% or greater yield for lipophilic phenolics. In certain embodiments, for example, the process achieves a recovery yield of 90% or greater yield for vitamins, 90% or greater yield for carotenoids, and 90% or greater yield for lipophilic phenolics.

The process of the invention may include a pre-treatment step, for example, to prepare the biomass to be more suitable for a particular extraction and/or separation techniques. For instance, the biomass feedstock (e.g., DDGS) may be ground to a desired state of particulates, preferably to a level suitable for efficient and effective extraction as well as separation with filtration and/or centrifugation. Other pre-treatment techniques include, for example, cutting, milling, pressing, shearing and chopping.

It is noted that for certain applications, it may be beneficial to conduct a repeat (e.g., a second or a third) round of extraction, separation and solvent removal to an extract product of the desired compositions. The repeat round may be identical to the previous round. The repeat round may also be different from the previous round in one or more aspects, for example, solvent choice and amount, length of extraction, techniques of residual biomass separation and removal of solvent.

It is noted that while the process may be generally performed as a batch process at different scales (e.g., from about 5 Kg to about 20 Kg, from about 20 Kg to about 200 Kg, at least 20 Kg of biomass per batch, at least 200 Kg of biomass per batch, at least 1,000 Kg of biomass per batch), the process may be designed as a continuous process whereby biomass feedstock is replenished continuously or periodically with a continuous extraction, residual separation and/or solvent removal.

In another aspect, the invention generally relates to a composition comprising one or more active ingredients extracted by a process disclosed herein.

Depending on the source of biomass, extraction conditions (e.g., solvent choice, solvent to biomass ratio, extraction temperature and length of time), method of separation and solvent removal, the compositions of the biomass extract may be varied. Thus, the biomass extract can be processed such as to result in certain compositions of active ingredients.

In yet another aspect, the invention generally relates to a biomass extract that includes, by weight: from about 0.01% to about 20% of vitamins; from about 0.01% to about 20% of flavonoids; from about 0.01% to about 20% of carotenoids; from about 0.01% to about 20% of tocopherols; from about 0.01% to about 30% of lipophilic phenolics, and from about 0.01% to about 30% of phenolic acids.

In certain embodiments, the biomass extract comprises, by weight: from about 0.01% to about 20% (e.g., from about 0.01% to about 20%, from 0.1% to about 20%, from about 1.0% to about 20%, from about 0.01% to about 10%, from about 0.01% to about 5.0%, from about 0.1% to about 10%, from about 1.0% to about 5.0%) of vitamin E and vitamin B; from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of flavonoid anthocyanin; from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of carotenoid, beta-carotene and lutein; from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of tocopherols: including alpha-, delta-, and gamma-tocopherols; and from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of lipophilic phenolics: ferulic acid esters and coumaric acid esters.

In certain embodiments, the biomass extract comprises, by weight: from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of vitamin E and vitamin B; from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of anthocyanin; from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of beta-carotene and lutein; from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of tocopherols: including alpha-, delta-, and gamma-tocopherols; and from about 0.01% to about 20% (e.g., from about 0.1% to about 20%, from about 1.0% to about 20%, from about 0.01% to about 10%, from about 0.01% to about 5.0%, from about 0.1% to about 10%, from about 1.0% to about 5.0%) of ferulic acid esters and coumaric acid esters.

In certain embodiments, the biomass extract comprises, by weight: from about 0.5% to about 20% (e.g., from about 1.0% to about 20%, from about 5.0% to about 20%, from about 0.5% to about 10%, from about 0.5% to about 5.0%, from about 1.0% to about 5.0%) of vitamin E and vitamin B; from about 0.5% to about 20% (e.g., from about 1.0% to about 20%, from about 5.0% to about 20%, from about 0.5% to about 10%, from about 0.5% to about 5.0%, from about 1.0% to about 5.0%) of anthocyanin; from about 0.5% to about 20% (e.g., from about 1.0% to about 20%, from about 5.0% to about 20%, from about 0.5% to about 10%, from about 0.5% to about 5.0%, from about 1.0% to about 5.0%) of beta-carotene and lutein; from about 0.5% to about 20% (e.g., from about 1.0% to about 20%, from about 5.0% to about 20%, from about 0.5% to about 10%, from about 0.5% to about 5.0%, from about 1.0% to about 5.0%) of tocopherols: including alpha-, delta-, and gamma-tocopherols; and from about 0.5% to about 20% (e.g., from about 1.0% to about 20%, from about 5.0% to about 20%, from about 0.5% to about 10%, from about 0.5% to about 5.0%, from about 1.0% to about 5.0%) of ferulic acid esters and coumaric acid esters.

In certain embodiments, the biomass extract comprises, by weight: from about 10% to about 20% of vitamin E and vitamin B; from about 10% to about 20% of anthocyanin; from about 10% to about 20% of beta-carotene and lutein; from about 10% to about 20% of tocopherols: including alpha-, delta-, and gamma-tocopherols; and from about 10% to about 20% of ferulic acid esters and coumaric acid esters.

In yet another aspect the invention generally related to a process for extraction of zein from a biomass.

In yet another aspect, the invention generally relates to a composition comprising zein produced by a process disclosed herein. In certain embodiments, a composition of the invention comprises from about 1% to about 90% (e.g., from about 1% to about 80%, from about 10% to about 80%, from about 20% to about 80%, from about 30% to about 80%, from about 40% to about 80%, from about 50% to about 90%, from about 60% to about 90%, from about 70% to about 90%, from about 80% to about 90%) of zein by weight.

In yet another aspect, the invention generally relates to a process for extracting nucleotides from a biomass. The process includes: (a) contacting the biomass with an alkaline aqueous solution under a condition and for a time sufficient to extract nucleic acids from the biomass, thereby giving rise to a remaining biomass and an alkaline aqueous phase comprising the extracted nucleic acids; (b) separating the remaining biomass and the alkaline aqueous phase comprising the extracted nucleic acids; (c) treating the alkaline aqueous phase comprising the extracted nucleic acids to precipitate nucleic acids from the aqueous phase; and (d) separating the precipitated nucleic acids from the aqueous phase to yield an extract composition comprising nucleic acids.

Any suitable biomass feedstock may be used. Exemplary biomass feedstock includes fermentation products of plants or plant-based materials. In certain embodiments, the biomass feedstock is fermentation products of various grains (e.g., corn, rice, wheat, barley, and rye). In certain preferred embodiments, the biomass feedstock is DDGS. In certain preferred embodiments, the biomass feedstock is DDGS produced from corn-based ethanol fermentation.

In certain embodiments, the biomass may include a spent biomass material from an alcoholic extraction of fermentation product of plants or plant-based materials, for example, a spent biomass material from an alcoholic extraction of fermentation product of grains selected from corn, rice, wheat, barley and rye. In certain embodiments, the biomass may include a spent biomass material generated from an alcoholic extraction of dried distillers grain with solubles (DDGS).

The biomass may be in any suitable form, for example, typically in particulate forms having sizes from about 0.5 nm to about 10 mm (e.g., from about 0.5 nm to about 8 mm, from about 0.5 nm to about 6 mm, from about 0.5 nm to about 5 mm, from about 0.5 nm to about 2 mm, from about 0.5 nm to about 1 mm, from about 1 nm to about 10 mm, from about 10 nm to about 10 mm, from about 50 nm to about 10 mm, from about 1 mm to about 10 mm).

The aqueous alkaline (i.e., basic) solution may have any suitable pH of greater than 7, for example, from about 8 to about 11. In certain embodiments, the aqueous alkaline solution has a pH from about 8 to about 9.5 (e.g., about 8.0, 8.2, 8.4, 8.6, 8.8, 9.0, 9.2, 9.4). In certain embodiments, the aqueous alkaline solution has a pH from about 9.5 to about 11 (e.g., about 9.6, 9.8, 10.0, 10.2, 10.4, 10.6, 10.8).

The aqueous alkaline solution may comprise any suitable solute to achieve the desired basic condition. For example, the solute may be selected from bases such as ammonia, sodium carbonate, sodium hydroxide, and potassium hydroxide.

The weight ratio of the aqueous alkaline solution to the biomass may be any suitable ratio, for example, from about 1:1 to about 20:1 (e.g., from about 1:1 to about 15:1, from about 1:1 to about 12:1, from about 1:1 to about 10:1, from about 1:1 to about 8:1, from about 2:1 to about 20:1, from about 5:1 to about 20:1, from about 8:1 to about 20:1, from about 10:1 to about 20:1, from about 5:1 to about 15:1, from about 5:1 to about 12:1, from about 5:1 to about 10:1, from about 7:1 to about 15:1, from about 7:1 to about 12:1, from about 7:1 to about 9:1).

For the step of separating the remaining biomass and the aqueous phase comprising nucleic acids, it may be carried out by any suitable technique, for example by filtration and/or centrifugation. The separation step may include a round of filtration or centrifuge or may include two or more rounds of filtration or centrifugation or a combination thereof.

In certain embodiments, separating the remaining biomass and the aqueous phase comprising nucleic acids is carried out by one or more rounds of filtration.

In certain embodiments, separating the remaining biomass and the aqueous phase comprising nucleic acids is carried out by one or more rounds of centrifuge.

The alkaline aqueous phase comprising nucleic acids is then treated to precipitate nucleic acids from the aqueous phase, for example, by adding one or more organic solvents (e.g., ethanol, ethyl acetate, and hexane) to the alkaline aqueous phase. In certain embodiments, an alcohol (e.g., ethanol) is added to the alkaline aqueous phase to precipitate nucleic acids from the aqueous phase. A co-organic solvent (e.g., another alcohol, ethyl acetate, and hexane) may be added simultaneous or sequentially.

The nucleic acids recovered in the biomass extract may include RNA molecules, DNA molecules or both, for example, yeast RNA and yeast DNA.

The process of the invention enables effective recovery of nucleic acids from the biomass feedstock. Actual yield of recovery of particular nucleic acids (RNAs and DNAs) depend on factors such as source of biomass, solvent pH, ratio to biomass, temperature and length of extraction, etc. The process can be designed to be suitable for extracting certain nucleic acid molecules, for example, preferably recover yeast RNA molecules.

In certain embodiments, the process achieves a recovery yield of 10% or greater (e.g., about 10% or greater, about 20% or greater, about 30% or greater, about 40% or greater, about 50% or greater, about 60% or greater, about 70% or greater, about 80% or greater, about 90% or greater) yield of nucleic acids present in the biomass prior to extraction.

The process of the invention may include a pre-treatment step, for example, to prepare the biomass to be more suitable for a particular extraction and/or separation techniques. For instance, the biomass feedstock (e.g., DDGS) may be ground to a desired state of particulates, preferably to a level suitable for efficient and effective extraction as well as separation with filtration and/or centrifugation. Other pre-treatment techniques include, for example, cutting, milling, pressing, shearing and chopping.

In certain embodiments, the biomass is pre-treated with one or more organic solvents prior to contacting the biomass with the alkaline aqueous solution.

Also, it may be beneficial to conduct a repeat (e.g., a second or a third) round of extraction by an aqueous alkaline solution, separation and solvent removal using the remaining biomass to achieve an extract product of the desired compositions. The repeat round may be identical to the previous round. The repeat round may also be different from the previous round in one or more aspects, for example, solvent choice and amount, length of extraction, techniques of residual biomass separation and removal of solvent.

It is noted that while the process may be generally performed as a batch process at different scales (e.g., at least 5 Kg of biomass per batch, at least 50 Kg of biomass per batch, at least 500 Kg of biomass per batch), the process may be designed as a continuous process whereby biomass feedstock is replenished continuously or periodically with a continuous extraction, residual separation and/or solvent removal.

In yet another aspect, the invention generally relates to a composition comprising nucleic acids produced by a process disclosed herein.

In certain embodiments of the biomass extract, the composition comprises from about 0.1% to about 90% (e.g., from about 0.1% to about 80%, from about 10% to about 80%, from about 20% to about 80%, from about 30% to about 80%, from about 40% to about 80%, from about 50% to about 90%, from about 60% to about 90%, from about 70% to about 90%, from about 80% to about 90%) of yeast RNA by weight. In certain embodiments of the biomass extract, the composition further comprises from about 0.1% to about 60% (e.g., from about 10% to about 60%, from about 20% to about 60%, from about 30% to about 60%, from about 40% to about 60%, from about 50% to about 60%, from about 0.1% to about 50%, from about 0.1% to about 40%, from about 0.1% to about 30%, from about 0.1% to about 20%, from about 0.1% to about 10%, less than 10%, less than 5%) of yeast DNA.

In certain embodiments, the weight ratio of yeast RNA to yeast DNA is from about 5:1 to about 20:1 (e.g., from about 5:1 to about 15:1, from about 5:1 to about 12:1, from about 5:1 to about 10:1, from about 5:1 to about 8:1, from about 8:1 to about 20:1, from about 10:1 to about 20:1, from about 12:1 to about 20:1, from about 15:1 to about 20:1, from about 6:1 to about 12:1, from about 7:1 to about 10:1).

The crude extract of nucleic acids may be further processed to covert them to nucleotides and/or further otherwise transformed as needed.

In certain embodiments, the process of the invention further includes: enzymatically hydrolyzing the separated nucleic acids to yield a mixture of 5′-nucleotide monophosphate (MP) monomers selected from GMP, UMP, AMP, and CMP.

In yet another aspect, the invention generally relates to a composition comprising 5′-nucleotide monophosphate monomers produced by a process herein.

In yet another aspect, the invention generally relates to a biomass extract, comprising by weight: from about 0.1% to about 50% of GMP; from about 0.1% to about 50% of UMP; and from about 0.1% to about 50% of CMP.

In certain embodiments, zein so produced is similar or substantially identical to zein obtained from a commercial source or extracted from corn.

### Examples

### Example I

### Extraction of Nucleic Acid from DDGS

Extraction of Nucleic Acid from DDGS (Method One)

DDGS was weighed (100.0 g) and placed in a blue cap bottle. To the solid, sodium chloride solution (8%, 300 mL) was added and the mixture was heated to 90° C. for 2 hours. The resulting slurry was cooled to 10° C. quickly and then filtered. The filtrate was adjusted to pH 2.5 with hydrochloric acid. The solution was kept for 12 hours at 4° C. refrigerator to precipitate RNA. The solution was then centrifuged and the precipitation was washed with anhydrous ethanol (50 mL) twice. The residue was dissolved in de-ionized water (50 mL) and filtered. The filtrate measured by UV-VIS spectroscopy. The results are shown in FIG. 3 and the concentrations of nucleic acid were estimated from the absorbance values at 260 nm and shown in Table 1.

TABLE 1
Estimated nucleic acid contents of distiller biomass

| | Estimated Extraction yields | Moisture | Yield (based on |
|---|---|---|---|
| Sample | based on wet weight | (%) | dry weight) |
| Yeast | 2.67 | NA (as ref.) | 2.67 |
| DDGS | 0.012 | 12.07 | 0.0136 |

Calculated based on the absorbance value at 260 nm (One absorbance unit equals to 40 μg/mL.

Extraction of Nucleic Acid from DDGS Diluted Base Method

In a flask (250 mL), water (180 mL), sodium hydroxide (2.0 g), and DDGS (20 g) were added. The resulting slurry was stirred for 30 minutes and the pH was adjusted to neutral (7.0) with hydrochloric acid (6.0 M) and stirred for ten minutes. The mixture was then heated to 90° C. for 10 minutes and cooled to room temperature then in 4° C. refrigerator overnight. The slurry was centrifuged at 3500 RPM and the supernatant was decanted. The supernatant was acidified with hydrochloric acid (6 M) to pH 2.50 and kept at 4° C. overnight. The resulting mixture was centrifuged at 6000 RPM and the precipitate was combined and washed with ethanol (95%, 10 mL) three times. The solid (crude nucleic acid) was dissolved in 1 liter distilled water. The absorbance of at 260 nm was measured to be 0.093. Based on this value, the percentage of RNA in DDGS is estimated to be 0.020%.

Extraction of RNA from DDGS Saline Method

In a flask (250 mL), water (180 mL), sodium chloride (20 g), and DDGS (20 g) were added. The mixture was then heated to 95° C. for two hours and cooled to room temperature before it was placed at 4° C. overnight. The slurry was centrifuged at 3500 RPM and the supernatant was decanted. The supernatant was acidified with hydrochloric acid (6 M) to pH 2.50 and kept at 4° C. overnight. The resulting mixture was centrifuged at 6000 RPM and the precipitate was combined and washed with ethanol (95%, 10 mL) three times. The solid (crude nucleic acid) was dissolved in 1 liter distilled water. The absorbance of at 260 nm was measured to be 0.295. Based on this value, the percentage of RNA in DDGS was estimated to be 0.059%.

Extraction of RNA from DDGS after Defatting with Ethanol

In a flask (250 mL) were added ethanol (95%, 100 mL) and DDGS (20 g) was stirred for 30 min. The mixture was filtered and the residue was placed in a flask and mixed with sodium chloride (20 g) and water (180 mL). The mixture was then heated to 95° C. for two hours and cooled to room temperature then kept at 4° C. overnight. The slurry was centrifuged at 3500 RPM and the supernatant was decanted. The supernatant was acidified with hydrochloric acid (6 M) to pH 2.50 and kept at 4° C. overnight. The resulting mixture was centrifuged at 6000 RPM and the precipitate was combined and washed with ethanol (95%, 10 mL) three times. The solid (crude nucleic acid) was dissolved in 1 liter distilled water. The absorbance of at 260 nm was measured to be 0.320. Based on this value, the percentage of RNA in DDGS was estimated to be 0.064%.

Hydrolysis of Nucleic Acid to Nucleotides:

RNA extract from DDGS (10 mg obtained from previous procedure 00106) was mixed with water (10 mL) and nuclease P1 from Penicillium citrinum lyophilized powder (0.2% of the weight of DDGS nucleic acid). The mixture was adjusted to pH 5.0 and heated at 60° C. for 8 hours. The resulting solution was heated at 90° C. for 15 min to deactivate the nuclease P1. The resulting solution was subjected to HPLC analysis (for nucleotides) and the results shown the GMP concentration in the solution is 10 mg/L and that of AMP is 5.5 mg/L.

### Example II

### Sequential Extraction of DDGS (Five-Kg Scale) into Different Fractions (Method One)

In this example, a sequential extraction of DDGS at 5-Kg scale is carried out involving: oil (1:1 ethyl acetate and ethanol mixture)->zein (70% ethanol)->nucleotides (water and 5′phosphodiesterase)->spent DDGS, as provided in more detail herein.

Extraction of Oil with Bioactives

To a 20 L reactor with heating jacket, absolute ethanol (7.5 L) and ethyl acetate (7.5 L) was added and mixed by stirring. To the mixture, DDGS (5 Kg) was added and heated from room temperature to 60° C. It took about 45 min. The mixture was kept at 60° C. with stirring for one hour and cooled. After 30 minutes, the temperature reached 30° C. The mixture was decanted and centrifuged. The solvents in the filtrate was recycled (12.6 L, 84%) by rotary evaporator to give oil 729.4 grams. The residue was placed in a reactor and mixed with ethyl acetate and ethanol mixture (1:1, 15 L). The mixture was heated to 60° C. after 45 min and kept stirring for one hour at the temperature. The mixture was cooled to 30° C. after 30 min and the slurry was decanted and filtered. The solvents from the filtrate was recycled (12.7 L, 85%) to give oil 179.2 grams. The total yield of oil with bioactives is 908.6 g (18.2%). The residue will be used for further extractions.

Analysis:

The HPLC of oil solutions were carried out on Waters 2695 HPLC system coupled with a photodiode array detector (PDA) (Waters 2996), an auto-sampler (Waters 717 plus). The HPLC column was a 250×4.6 mm, 5 μm RP C18 column (Waters, Atlantis T3). The mobile phase consisted of A (0.04% acetic acid in deionized water) and B (0.04% acetic acid in methanol). The gradient procedure for HPLC separation is shown in Table 2.

TABLE 2
Gradient Procedure for Chromatographic Separation

| | | Phase composition | |
|---|---|---|---|
| Time/min | Flow rate mL/min | A/% | B % |
| 0 | 1 | 100 | 0 |
| 1 | 1 | 100 | 0 |
| 8 | 1 | 90 | 10 |
| 24 | 1 | 75 | 25 |
| 34 | 1 | 55 | 45 |
| 45 | 1 | 45 | 55 |
| 60 | 1 | 0 | 100 |
| 90 | 1 | 0 | 100 |
| 95 | 1 | 100 | 0 |
| 105 | 1 | 100 | 0 |

Identification of phenolic acid (vanillic, caffeic, p-coumaric, and ferulic acid), lutein, and α-tocopherol were based on comparing the retention time and UV absorbance of the respective compounds. The concentrations of the key compounds are: vanillic acid 8.74 mg/100 g oil); caffeic acid, 8.68 mg/100 g oil; p-coumaric acid, 14.49 mg/100 g oil, ferulic acid 16.38 mg/100 g oil, lutein, 31.62 mg/100 g oil; α-tocopherol, 40.12 mg/100 g oil. Typical HPLC finger print is shown in FIG. 4.

### Zein

The residue from the extraction of oil with bioactive (from above) was placed in the 20 L reactor and mixed with 70% ethanol (15 L) and heated to 60° C., it took 50 minutes to raise the temperature to 60° C. The slurry was stirred for 1 hour and cooled down to 30° C. after 35 minutes. The slurry was decanted and centrifuged. The solvents in the filtrate were recycled by rotary evaporator (12.4 L, ethanol concentration 75%). The residue was dried in vacuum for 12 hours to give solid 348.4 g. The residue was placed in 20 L reactor and mixed with 70% ethanol (15 L). The mixture was heated to 60° C. in 50 min and kept stirring for one hour under that temperature. The mixture was cooled to 30° C. in 35 minutes before it is decanted and centrifuged. The filtrate was subjected to rotary evaporation to recycle solvents (12.4 L, 75% ethanol) and resulted 203.5 g solid after drying in 60° C. vacuum oven for ten hours. Total yield of the zein is 551.7 g (11%).

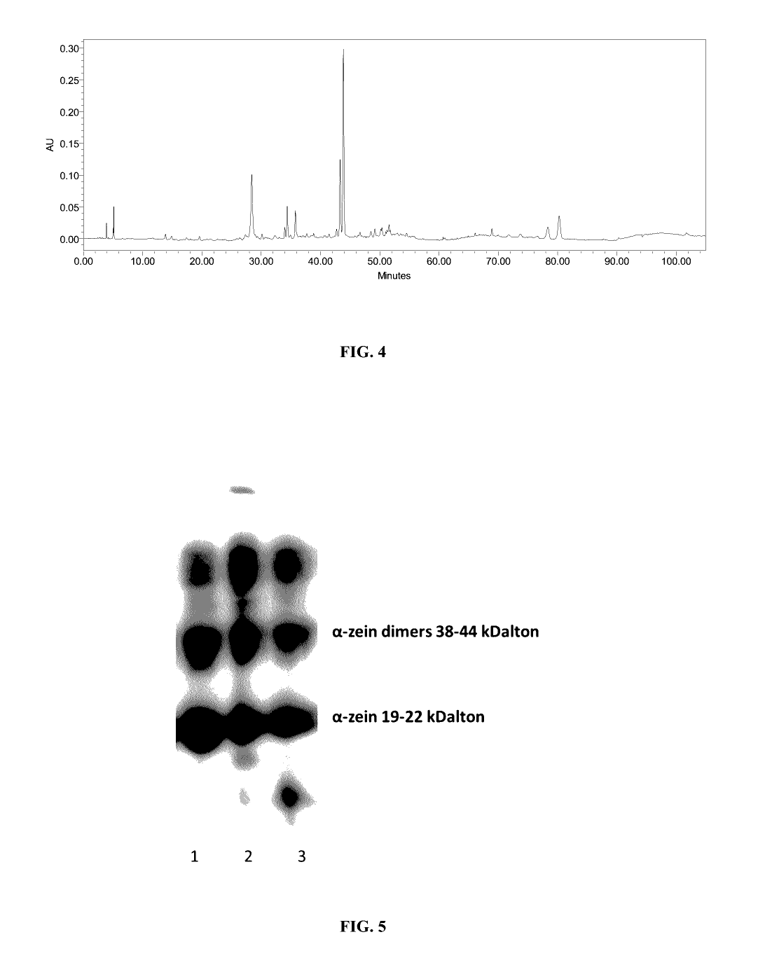

Analytical method for zein profile of DDGS in comparison with commercial zein and that extracted from corn. The electrophoresis of zein was operated with electrophoresis apparatus from Bio-rad Company (Hercules, Calif., USA). The molecular weight profile of extracted zein with a 4% stacking gel and 12% separating gel in an SDS-Tris-Glycine buffer system, following SDS-PAGE method for zein by Paraman (Paraman, I.; Lamsal, B. P., Recovery and characterization of α-zein from corn fermentation coproducts. Journal of Agricultural and Food Chemistry 2011, 59, 3071.). Briefly, the zein solutions were diluted to 10 g/L by a sample buffer: 125 mM Tris-HCl at pH 7.0, 2% SDS, 10% glycerol, 5% 2-mercaptoethanol and 0.05% bromophenol blue. The protein solutions were centrifuged to remove the precipitation, and 15 pt of the solution was loaded on to the gel. Electrophoresis was performed at 200 V for 60 min. The gel was stained by 0.1% Coomassie brilliant blue solution. Bio-rad molecular weight marker ranging from 10 to 200 kDa (Hercules, Calif., USA) was used. The selective image of the zeins is shown in FIG. 5. As it can be seen from the FIG. 5, the DDGS zein shows comparable to that of the zein from commercial source and that of zein extracted from corn.

### Nucleotides

In the 20 L reactor, the residue from extraction of zein (from above) was mixed with water (15 L) and heated to 60° C. before 50 grams of 5-phosphordiesterase (from Nuclease P1 from Penicillium citrinum, 50 g) was added. The mixture was stirred at 60° C. for 24 hours and cooled to 40° C. in 30 min. The slurry was centrifuged at 3000 r/min for 5 min. The filtrate (about 13 liters) was filtered again to remove small amount of white precipitate. The resulting clear filtrate was spray dried. It took about 12 h complete the drying process, which yielded light yellow powder 288 grams (5.6%) of nucleotide fraction.

Analysis of Nucleotide Contents:

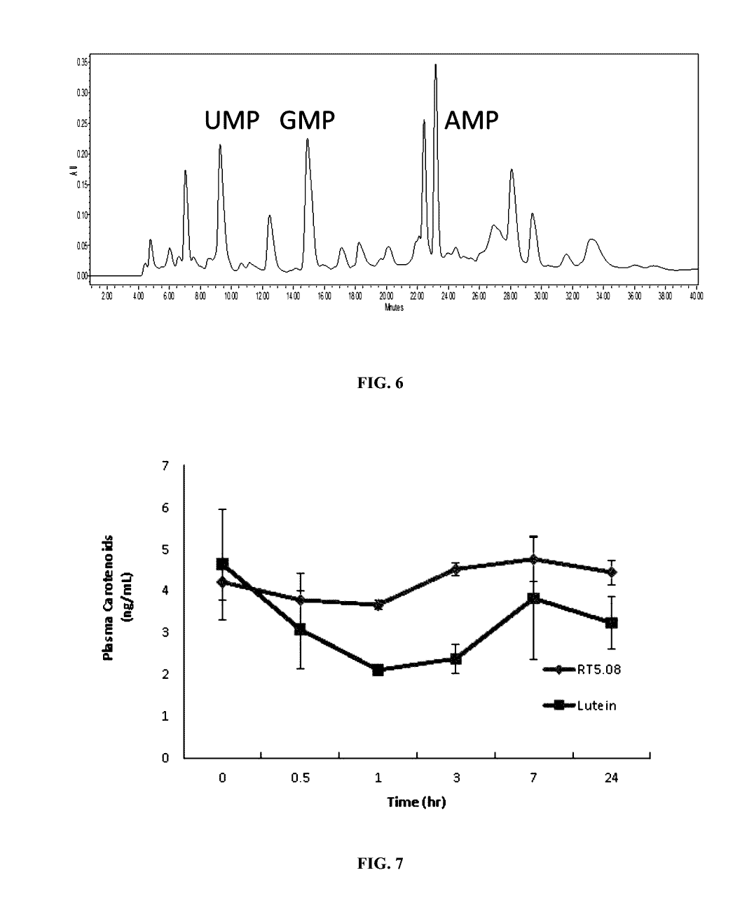

The HPLC analysis was carried out on a Waters 2695 HPLC system coupled with a photodiode array detector (PDA) (Waters 2996) and auto sampler (Waters 717 plus). The stationery phase was a HPLC column was a 250×4.6 mm, 5 μm C18 column (Atlantis, Waters). The mobile phase A (K₂HPO₄, 0.1 M, pH=5.6) was made by dissolving 13.6 g K₂HPO₄ in 1000 mL of de-ionized water and adjusting the pH to 5.6 with 2 M KOH solution. Mobile phase B was 100% of methanol. The solvent gradient sequence was shown in Table 3. HPLC chromatogram of nucleotide extracts is showed in FIG. 6.

TABLE 3
Gradient procedure for nucleotides HPLC analysis

| | | Phase composition | |
|---|---|---|---|
| Time (min) | Flow rate (mL/min) | % A | % B |
| 0 | 0.5 | 100 | 0 |
| 5 | 0.5 | 100 | 0 |
| 14 | 0.5 | 90 | 10 |
| 15 | 0.5 | 80 | 20 |
| 35 | 0.5 | 80 | 20 |
| 36 | 0.5 | 100 | 0 |
| 50 | 0.5 | 100 | 0 |

### Spent DDGS

The residue from nucleotide extraction (from above) was washed with small amount of water. The total water used to wash the residue was 1.5 L. The residue was place in oven and dried at 100° C. for 2 days to give spent DDGS 3.00 Kg (yield 60%) solid.

HPLC Quantification of Amino Acid Profile of Spent DDGS:

The HPLC analysis of amino acids was followed the standard method of Waters: AccQTag. The AccQTag Derivatization Kit and AccQTag Eluent A were bought from Waters (Milford, Mass., USA). The mobile phase A consisted of 50 mL of AccQTag Eluent A concentrate and 500 mL DI water and the mobile phase B was acetonitrile, and the mobile phase C was di-ionized water. The hydrolysate was filtered by a 0.45 μm micro-filter and derived. The derivatization procedures were followed Waters: 70 pt buffer and 20 μL derivatization reagent were added to 10 pt of hydrolysate. The mixture was shaken for 15 seconds before putting in a block heater for 10 min at 55° C.

TABLE 4
Amino Acids

| | Concentration |
|---|---|
| Amino acids | (mg/g spent DDGS) |
| Asp | 5.37 |
| Ser | 3.49 |
| Glu | 14.93 |
| Gly | 2.90 |
| His | 2.49 |
| Arg | 3.59 |
| Thr | 2.84 |
| Ala | 7.36 |
| Pro | 6.79 |
| Cys | <1 |
| Tyr | 3.55 |
| Val | 4.34 |
| Met | 1.13 |
| Lys | 1.97 |
| Ile | 4.03 |
| Leu | 9.51 |
| Phe | 4.54 |
| Trp | <1 |

Total amino acid: 78.8 mg/g spent DDGS.

In summary, product yields are shown in the following Table 5.

TABLE 5
Yields of products from DDGS refinery at 5 Kg scale

| | | Solvents |
|---|---|---|
| Fractions | Yield (%) | recycle rate (%) |
| Oil with bioactives | 908.6 g (18.2%) | 84% |
| Zein | 551.7 g (11%) | 88% |
| Nucleotides | 288 g (5.6%) | n/a (water) |
| Spent DDGS | 3.00 Kg (60%) | n/a |
| Total solids recovered | 4.758 Kg | |

### Example III

### Extraction of DDGS (5-Kg Scale) (Method Two)

### Nucleotides

To a jacketed reactor (20 L), water (17 L) was added and stirred at 100 rpm followed by DDGS (5 kg). The mixture was rather viscous. After the addition of DDGS, the reactor was heated to 60° C. through the heat circulator, which took 35 minutes. To the solution, 5-phosphodiesterase (from Nuclease P1 from Penicillium citrinum, 50 g) was added and stirred for 24 hours. The slurry was centrifuged to give cloudy filtrate, which was centrifuged again to give 13.7 L liquid. Spray drying of the liquid yielded 740 grams of brown viscous solid, which is the nucleotides fraction.

### Zein

To the jacketed reactor ethanol (70%, 15 L) was added along with the residue from above operation and stirred at 60° C. for one hour. After the temperature was cooled to 30° C., the mixture was dispensed from the reactor and centrifuged to separate the residue 2 and the filtrate. After evaporation of the volatiles from the filtrate, viscous solid was obtained with yield of 297 grams zein after vacuum drying at 70° C. for ten hours. The step was repeated to give 213 gram more solids. The total yield for zein is 511 g.

Oil with Bioactives

To a jacketed reactor (20 L) ethyl acetate and ethanol (1:1) was place and stirred. To the mixture, the residue 2 was added to the mixture and heated to 60° C. for one hour. After the temperature of the reaction mixture was cooled to 30° C., the mixture was dispensed from the reactor and centrifuged. The filtrate was collected and the residue was extracted again using the same amount of solvent (20 L), ethyl acetate and ethanol (1:1) and filtered to give residue 3 and filtrate. After evaporation of the solvents, the resulting oil fraction yield was 240 g.

### Spent DDGS

The residue 3 was vacuum dried to give 3174 g of solid, which is the spent DDGS (dark brown color).

In total, extraction of 5 Kg DDGS yielded:

3174 g of spent DDGS (63.5%)

240 g oil (4.8%)

511 g zein (11%)

740 g nucleotides (14.8%)

The total weight of the products is: 4665 grams (recycling rate of 93.3%).

### Example IV

### Bioavailability Studies on Phytochemicals Extracted from DDGS

Animal Study.

Thirty-six male CD-1 mice (22-24 g) were purchased from Charles River Labs (Wilmington, Mass.). Before the study, they were allowed acclimation for at least 3 days in an SPF facility. Animal housing, handling and procedures were conducted under the protocols or guidelines approved by the Institutional Animal Care and Use Committee (IACUC) of Cephrim Biosciences, Inc. (Woburn, Mass.). Three mice were housed in each cage. The temperature, humidity and light/dark cycle were well maintained at 68-76° F., 40-60% relative humidity, with a 12 h light/dark cycle. Mice were allowed free access to water and food.

On the first day of the study, the mice were randomly assigned into two groups, alcohol extract (AE, of DDGS) group and oil extract (OE, of DDGS) group. Each group of mice was further divided into 6 subgroups, representing 6 time points (i.e., 0 hr, 0.5 hr, 1.0 hr, 3.0 hr, 7.0 hr and 24 hr). Each of two extracts was given to each group of mice by oral gavage at the dosage of 2 mL for AE and 2 mL for OE. The time of dosing was set as the Time Zero. Blood samples were collected by cardiac puncture and were immediately transferred to a set of heparinized tubes.

Blood samples at Time-0 hr were collected right before dosing. And at each of rest 5 time points the Time-0.5 hr, -1.0 hr, -3.0 hr, -7.0 hr and −24 hr after the extracts were given, blood samples were collected from each subgroup (n=3). All blood samples were placed on ice after their collection and were spun at 14000 rpm/min. The top plasma were transferred into new pre-labeled tubes and the samples were stored at −70° C. until analysis.

Determination of Tocopherols and Carotenoids in Plasma.

Mice plasmas (150 pt) were mixed with 600 pt of hexane and shaken at 200 rpm for 10 min. The mixtures were centrifuged at 14,000 rpm for 6 min. The supernatant was evaporated to dryness under nitrogen and replaced with 100 pt of methanol. The extract was then centrifuged at 14,000 rpm for 5 min and injected into HPLC.

HPLC Conditions for Tocopherols:

Compounds were separated on a C30 reverse-phase column (250×4.6 mm, 5 um) at a flow rate of 1 mL/min. Methanol was used as mobile phase. The column was kept at 6° C. Total run time is 35 min. The detection was conducted in a fluorescence detector with excitation of 292 nm and emission of 336 nm.

HPLC Conditions for Carotenoids:

Compounds were separated on a Develosh Rpaqueous C30 reverse phase column (250×4.6 mm, 5 μm) at a flow rate of 1 mL/min. The HPLC separation was accomplished using a two-solvent gradient system. The mobile phases consisted of A) methanol:MTBE:1% ammonium acetate (83:15:2, V/V/V) and B) methanol:MTBE:1% ammonium acetate (8:90:2, V/V/V). The column was kept at 16° C. Total run time is 36 min (post run 5 min). The detection was at 450 nm.

Determination of Phenolics in Plasma.

Mice plasmas (200 pt) were mixed with 12 μL of 10% ascorbic acid-40 mM KH2PO4-0.1% EDTA, 30 μl of 50 mM potassium phosphate (pH 7.4), 350 units of β-d-glucuronidase type X-A from E. coli (Sigma Chemical Co, St. Louis, Mo., USA) and 6 units of sulfatase type VIII from abalone entrails (Sigma Chemical Co, St. Louis, Mo., USA). The mixture was incubated at 37° C. for 45 min. The reaction was stopped by the addition of 2 mL of ethyl acetate followed by vigorous shaking for 20 min and centrifugation at 4° C. at 2000×g for 5 min. The supernatant was transferred to a clean tube, and the ethyl acetate extraction was repeated. 10 pt of 0.02% ascorbic acid:0.005% EDTA was added to the pooled supernatant fraction and vortexed thoroughly to mix. The supernatant was then evaporated to dryness under nitrogen at room temperature. The samples were reconstituted in 100 μL of methanol, vortexed well, sonicated for 10 min, and centrifuged (14,000 rpm, 5 min).

HPLC Conditions for Phenolics:

Compounds were separated on a Phenomenex C18 phenyl-hexyl column (250×4.6 mm, 5 μm) at a flow rate of 1 mL/min. The HPLC separation was accomplished using a two-solvent gradient system. The mobile phases consisted of A) water:acetic acid:acetonitrile (89:2:9, v/v/v) with addition of 10 mM PBS (pH 3.4) and B) 80% acetonitrile (v/v) with addition of 1 mM PBS (pH 5). The column was kept at 20° C. Total run time is 50 min (post run 5 min). The detection was achieved using an ESA 5600 CoulArrray electrochemical detector with potential settings at 500 and 800 mV.

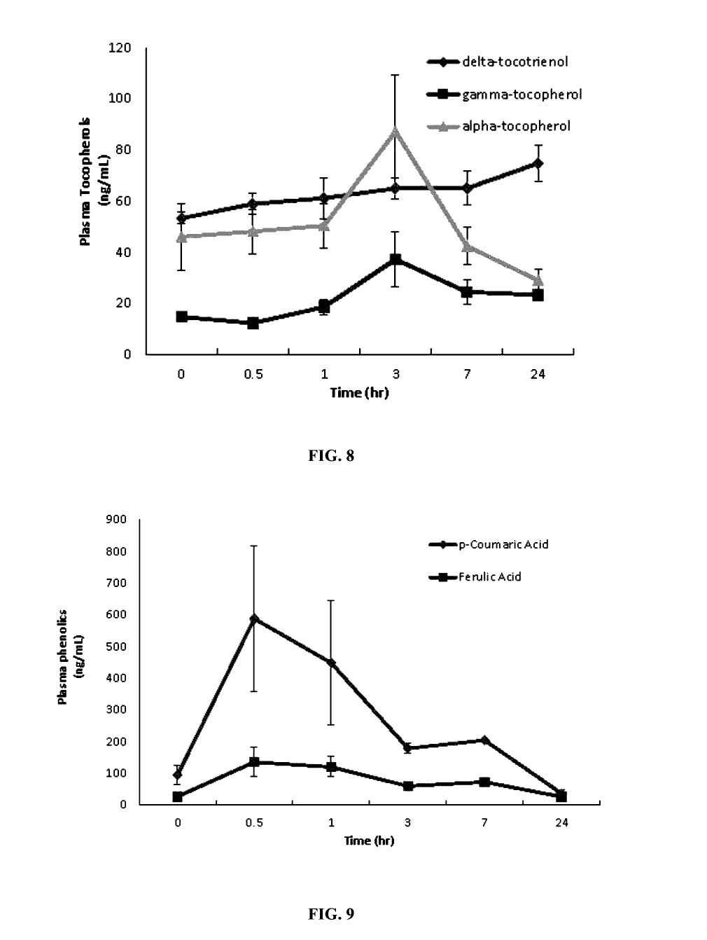

Results of bioavailability tests are provided in FIGS. 7-9. FIG. 7 shows that maximal absorption of carotenoids in plasma of mice was found after 7 hours of gastric infusion. In FIG. 8, maximal absorption of gamma- and alpha-tocopherols in plasma of mice was found after 3 hours of gastric infusion, while maximal absorption of delta-tocotrienol in plasma of mice was found after 24 hours of gastric infusion. FIG. 9 shows that maximal absorption of phenolics in plasma of mice was found after 0.5 hours of gastric infusion. The phytochemicals identified in DDGS were found to be bioavailable as demonstrated in this mice study.

In this specification and the appended claims, the singular forms “a,” “an,” and “the” include plural reference, unless the context clearly dictates otherwise. Unless defined otherwise, all technical and scientific terms used herein have the same meaning as commonly understood by one of ordinary skill in the art. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A process for extracting one or more active ingredients from a biomass, comprising:
contacting the biomass with a solvent A under a condition and for a time sufficient to extract the one or more active ingredients from the biomass into the solvent A, thereby giving rise to a residual biomass I and a liquid phase comprising the solvent A and the extracted one or more active ingredients;
separating the residual biomass I and the liquid phase comprising the solvent A and the extracted one or more active ingredients; and
removing the solvent A from the liquid phase to yield an extract composition comprising the one or more active ingredients as a concentrated mixture or in substantially pure forms.

2. The process of claim 1, further comprising:
contacting the residual biomass I with a solvent B to extract proteins into the solvent B, thereby giving rise to a residual biomass II and a liquid phase comprising the solvent B and proteins; and
separating the residual biomass II and the liquid phase comprising the solvent B and the extracted proteins; and removing the solvent B from the liquid phase to yield an extract composition comprising proteins.

3. The process of claim 2, further comprising:
contacting the residual biomass II with a solvent C and an enzyme to extract nucleotides into the solvent; and
separating the residual biomass II and the liquid phase comprising the solvent C and the extracted nucleotides; and removing the solvent C from the liquid phase to yield an extract composition comprising nucleotides.

4. The process of claim 1, wherein the biomass comprises a fermentation product of plants or plant-based materials.

5. The process of claim 1, wherein the biomass comprises a fermentation product of grains selected from corn, rice, wheat, barley and rye.

6. The process of claim 1, wherein the biomass comprises dried distillers grain with solubles (DDGS).

7. The process of claim 1, wherein the biomass comprises a spent biomass material from an alkaline aqueous extraction of fermentation product of plants or plant-based materials.

8. The process of claim 7, wherein the spent biomass material is from an alkaline aqueous extraction of fermentation product of grains selected from corn, rice, wheat, barley and rye.

9. The process of claim 7, wherein the spent biomass material is from an alkaline aqueous extraction of dried distillers grain with solubles (DDGS).

10. The process of claim 1, wherein the biomass is in the form of particles having the sizes from about 0.5 μm to about 10 μm.

11. (canceled)

12. The process of claim 1, wherein the solvent A comprises an alcohol.

13. (canceled)

14. (canceled)

15. The process of claim 1, wherein the solvent A comprises a first co-solvent and a second co-solvent.

16. The process of claim 15, wherein the first co-solvent is an alcohol and the second co-solvent is selected from another alcohol, water, acetone, ethyl acetate, and hexanes.

17-21. (canceled)

22. The process of claim 1, wherein separating the residual biomass and the liquid phase comprising the solvent A and the one or more active ingredients is carried out by one or more of: filtration and centrifuge.

23. (canceled)

24. (canceled)

25. The process of claim 1, wherein removing the solvent A from the liquid phase comprises removing the solvent by evaporation.

26. The process of claim 25, wherein removing the solvent A from the liquid phase comprises removing substantially all of the solvent A by evaporation under one or more of: a raised temperature and a reduced pressure.

27. (canceled)

28. The process of claim 1, wherein the removed solvent A from the liquid phase is recycled.

29. The process of claim 1, wherein the one or more active ingredients are selected from vitamins, flavonoids, carotenoids, tocopherols, and lipophilic phenolics, phenolic acids.

30-34. (canceled)

35. The process of claim 1, wherein the process achieves a recovery yield of one or more of: 1% or greater yield for vitamins, 1% or greater yield for carotenoids, and 1% or greater yield for lipophilic phenolics.

36-39. (canceled)

40. The process of claim 1, wherein the process is a batch process.

41-43. (canceled)

44. The process of claim 1, wherein the process is a continuous process.

45. A composition comprising one or more active ingredients extracted by a process according to claim 1.

46. A biomass extract comprising, by weight:
from about 0.01% to about 20% of vitamins;
from about 0.01% to about 20% of flavonoids;
from about 0.01% to about 20% of carotenoids;
from about 0.01% to about 20% of tocopherols; and
from about 0.01% to about 30% of lipophilic phenolics and phenolic acids.

47-49. (canceled)

50. A process for extracting nucleotides from a biomass, comprising:
contacting the biomass with an alkaline aqueous solution under a condition and for a time sufficient to extract nucleic acids from the biomass, thereby giving rise to a remaining biomass and an alkaline aqueous phase comprising the extracted nucleic acids;
separating the remaining biomass and the alkaline aqueous phase comprising the extracted nucleic acids;
treating the alkaline aqueous phase comprising the extracted nucleic acids to precipitate nucleic acids from the aqueous phase; and
separating the precipitated nucleic acids from the aqueous phase to yield an extract composition comprising nucleic acids.

51-80. (canceled)

81. The process of claim 50, further comprising:
enzymatically hydrolyzing the separated nucleic acids to yield a mixture of 5′-nucleotide monophosphate monomers selected from GMP, UMP, AMP, and CMP.

82. A composition comprising 5′-nucleotide monophosphate monomers produced by a process according to claim 81.

83. The composition of claim 82, comprising by weight:
from about 0.1% to about 50% of GMP;
from about 0.1% to about 50% of UMP;
from about 0.1% to about 50% of AMP; and
from about 0.1% to about 50% of CMP.
